(19) United States
(12) Patent Application Publication
(10) Pub. No.: US 2008/0058211 A1
(43) Pub. Date: Mar. 6, 2008

(54) Method and Composition for the Modulation of Toxin Resistance in Plant Cells

(75) Inventor: Stanley J. Roux, Austin, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 11/837,173

(22) Filed: Aug. 10, 2007

Related U.S. Application Data
(60) Provisional application No. 60/837,417, filed on Aug. 11, 2006.

(51) Int. Cl. A01N 57/16; A01H 1/00; A01P 13/00; C07H 21/00; C12N 5/04; C12N 5/10

(52) U.S. Cl. 504/197; 435/419; 536/23.1; 800/278

(57) ABSTRACT

The present invention includes a methods and compositions that modulate drug resistance in a plant through the addition of one or more extracellular nucleotides that contact a plant cell.

### CROSS-REFERENCE TO RELATED APPLICATIONS

This application is a related to U.S. patent application Ser. No. 10/134,019, filed Apr. 25, 2002 and U.S. patent application Ser. No. 10/047,251, filed Jan. 14, 2002, the contents of which are incorporated by reference herein in their entireties. This application claims priority to U.S. Provisional Patent Application Ser. No. 60/837,417 filed Aug. 11, 2006.

The U.S. Government may own certain rights in this invention pursuant to the terms of the NIH grant number IBN-0344221. Without limiting the scope of the invention, its background is described in connection with extracellular signaling methods and compositions, as an example.

### TECHNICAL FIELD OF THE INVENTION

The present invention relates in general to modulating drug resistance pathways in plants, and more particularly, modulating extracellular nucleotide concentrations to affect drug resistance pathways in plant cells to modulate the plants resistance to certain herbicides.

### BACKGROUND OF THE INVENTION

Although ATP functions inside the cells as the principal energy currency of the cell, extracellular ATP functions as an agonist that does not have to be hydrolyzed to activate responses in cells [1]. This extracellular ATP function was restricted to studies of animal cells, where adenine nucleoside triphosphates and diphosphates mediate a wide variety of biological processes in the extracellular matrix (ECM) at specialized receptors known as P2-purinoceptors.

Extracellular ATP (eATP) has been reported to have numerous effects on the physiology of plants, e.g., altering both developmental programs and responses to environmental stimuli. Early studies showed that exogenous application of ATP could induce the closure of the Venus Fly Trap [2], affect cytoplasmic streaming in Chara cells [3], modulate stomatal aperture in Commelina communis [4] and stimulate pollen tube generative nuclear divisions in Lilium lingiflorum [5]. Most of these reports indicated that the applied ATP was somehow, directly or indirectly, altering the energy charge of the cell, and thus was still playing its standard role of driving energy-dependent reactions.

### SUMMARY OF THE INVENTION

The present inventors recognized a need for a method and composition for modulating extracellular nucleotide concentrations in order to affect the drug resistance pathways of cells to modulate resistance to certain drug molecules in these cells. The present inventors recognized that extracellular nucleotides (eNTP) (e.g., ATP, ADP, UTP, UDP, CTP, CDP, TTP, TDP, GTP, GDP, dATP, dADP, dUTP, dUDP, dCTP, dCDP, dTTP, dTDP, dGTP, dGDP and stable analogues) had been reported to have numerous effects on the physiology of plants, altering both developmental programs and responses to environmental stimuli. The present inventors recognized eNTP can act as an agonist, exerting its effects through interaction with cell surface receptors, and it is not necessary for the eNTP to undergo hydrolysis.

Higher plants exhibit cellular responsiveness to the exogenous applications of nucleotides in a manner consistent with a cell-cell signaling function for these molecules. Like animals, plants respond to extracellular ATP, ADP, and stable analogues (e.g., ATPγS and ADPβS) by increasing the cytoplasmic concentration of calcium. Agonist substrate specificity and concentration dependency suggest receptor mediation of these events, and pharmacological analysis points to the involvement of a plasma membrane-localized calcium channel. Extracellular ATP can also induce the production of reactive oxygen species and stimulate an increase in the mRNA levels of a number of stress and calcium regulated genes, suggesting a role for nucleotide-based signaling in plant wound and defense responses. Furthermore, the growth and development of plants can also be altered by the application of external ATP. Plant signaling networks represent a complicated series of interactions to affect plant physiological processes that are activated in response to extracellular ATP.

The invention disclosed herein, includes a method and composition to modulate the resistance of cells to certain drug molecules through the modulation of extracellular nucleotide concentrations. For example, the present invention provides a method of increasing superoxide and nitric oxide concentrations within a plant cell by increasing the extracellular concentration of the one or more nucleotides in the extracellular matrix of the plant cell. The one or more nucleotides activate one or more agents that increase the superoxide concentration.

The present invention provides a composition that modulates drug resistance in a plant. The composition includes one or more extracellular nucleotides that contact a plant cell and modulate drug resistance in the plant cell. The one or more extracellular nucleotides may be deoxyribonucleic acids or ribonucleic acids and include ATP, ADP, UTP, UDP, CTP, CDP, TTP, TDP, GTP, GDP, dATP, dADP, dUTP, dUDP, dCTP, dCDP, dTTP, dTDP, dGTP, dGDP, stable analogues and combinations thereof. The composition may modulate the drug resistance in a plant by affecting the concentration of one or more reactive oxygen species, NO or both within the plant cell. The composition may modulate the drug resistance in a plant by affecting the transcription of one or more genes, e.g., a ERF2 gene, a ERF3 gene, a ERF4 gene, a PAL1 gene, a LOX2 gene and a ACS6 gene.

The present invention also provides a composition that modulates drug resistance in a plant having one or more herbicides and one or more extracellular nucleotides that contact a plant cell to affect drug resistance of the plant to the one or more herbicides.

The present invention also provides a method of altering the resistance of a plant to a herbicide by increasing the concentration of one or more extracellular nucleotides about a plant cell to modulate drug resistance of the plant. A method of increasing the herbicidal sensitivity of a plant by increasing the concentration of one or more extracellular nucleotides about the plant cell to affect the herbicidal sensitivity of the plant is also provided.

The present invention also provides an herbicide potentiator that modulates drug resistance in a plant. The herbicide potentiator includes one or more extracellular nucleotides that contact a plant cell membrane and modulate drug resistance.

The present invention provides a method of activating one or more stress related biosynthetic genes or enhancing an ectophosphatase inhibitor activity in plant cells by increasing the concentration of one or more extracellular nucleotides about a plant cell. The one or more extracellular nucleotides activate one or more agents that increase transcription of the one or more stress related biosynthetic genes in the plant cell.

Another embodiment of the present invention is a method for modulating plant growth by adding of one or more extracellular nucleotides to a plant cell, wherein the extracellular concentration of the one or more nucleotides is different across a plant cell membrane and the contacting results in the activation of one or more agents that increase the superoxide concentration and one or more stress related biosynthetic genes transcripts.

The present invention also includes a method of modulating plant growth by contacting one or more extracellular nucleotides to a plant cell, wherein the extracellular concentration of the one or more nucleotides is different across a plant cell membrane.

### BRIEF DESCRIPTION OF THE DRAWINGS

For a more complete understanding of the features and advantages of the present invention, reference is now made to the detailed description of the invention along with the accompanying figure and in which:

FIG. 1 is a schematic of the nitric oxide signaling pathway with chemical mediators.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The terminology used and specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “potentiator” refers to a compound or agent that accentuates, enhances or potentiates an activity upon a cell so that the combined effect is greater than the sum of the effects of each one alone. The potentiator may be added, admixed, co-administered, administered in series or in parallel in conjunction with an optimal or sub-optimal dose of herbicide, another potentiator and/or another chemical agent. The potentiator may be added before, during or after a dose of an optimal or sub-optimal doses of herbicide, another potentiator and/or another agent and may even be conjugated directly with the one or more agents, either covalently or ionically. The present invention may be used in conjunction with numerous herbicides (e.g., herbicides which resemble established drugs implicated in multidrug resistance), plant hormones (e.g., cytokinin, auxins, gibberellins and brassinosteroids) and chemicals known to the skilled artisan.

Generally, the present invention provides a method of activating one or more stress related biosynthetic genes in plant cells by increasing the concentration of one or more extracellular nucleotides about a plant cell. The one or more extracellular nucleotides activate one or more agents that increase transcription of the one or more stress related biosynthetic genes in the plant cell.

The one or more extracellular nucleotides include ATP, ADP, UTP, UDP, CTP, CDP, TTP, TDP, GTP, GDP, dATP, dADP, dUTP, dUDP, dCTP, dCDP, dTTP, dTDP, dGTP and dGDP, which including stable analogues, modifications and combinations thereof. The modified and synthetic nucleotides are known to the skilled artisan and include modification to the base, the linkage or both. In addition, the modifications to the extracellular nucleotides may extend the activity of the nucleotide by maintaining the extracellular concentration of the nucleotides by increasing the resistance to degradation by extracellular enzymes.

The concentration of one or more extracellular nucleotides may be altered by a variety of mechanisms known to the skilled artisan, e.g., the extracellular addition of one or more extracellular nucleotides or nucleotides analogs. In addition, the concentration of the extracellular nucleotides may be increased as a result of the activity of another agent that triggers the release of extracellular nucleotides.

As a result of the increase in extracellular nucleotides, the transcription of an ERF2 gene, an ERF3 gene, an ERF4 gene, a PAL1 gene, a LOX2 gene, an ACS6 gene and combinations thereof may be increased. This may occur directly or through a chain reaction or signaling pathway of one or more agents, which cascade to result in the increase of transcription. For example, the increase in extracellular nucleotides results in the increase of reactive oxygen species and NO, which in turn alter transcription in the plant cell.

The present invention also provides method for modulating plant growth by adding of one or more extracellular nucleotides to a plant cell resulting in the extracellular concentration of the one or more nucleotides difference across a plant cell membrane. More recent studies have revealed that the hydrolysis of eATP is not required to induce responses in plant cells [6]. eATP plays a role as an agonist, exerting its effects through interaction with cell surface receptors, similar to what happens in animal cells. Some studies have shown that extracellular adenine nucleoside tri- and diphosphates mediate a wide variety of biological processes in the extracellular matrix (ECM) at specialized receptors known as P2-purinoceptors. The main types of these multigene family receptors in animals are P2X, 2-pass transmembrane (TM) subunits which oligomerize to form ligand-gated ion channels, and P2Y, 7-pass TM heterotrimeric G-protein linked receptors. The binding of ATP or ADP (among other NTPs and NDPs) activates these receptors, initiating secondary messenger systems and downstream signaling cascades, thereby affecting changes in gene expression and culminating in the induction of cell-type specific responses.

The first recognized physiological activity of these signaling agents was that of a co-neurotransmitter, and so this type of signaling was originally known as purinergic transmission [7]. Derivatives of ATP, including ADP and adenosine were also shown to have biological effects. For example, adenosine functions as a negative regulator of neurotransmitter release at specialized P1 purinceptors, functioning together with ATP to modulate smooth muscle contraction; and ADP signals blood platelet aggregation during thrombosis [8] and [9].

Rapid responses of plants to applied nucleotides: Induced changes in [Ca²⁺]_{cyt}. For example, in animal cells, activation of purinoceptors by extracellular nucleotides rapidly leads to changes in membrane potential and increases in the concentration of cytoplasmic calcium ions ([Ca²⁻]_{cyt}). These nucleotides alter membrane transport properties. The effects of these proteins are often assayed to examine the signaling roles of eATP and eADP in plants.

The rapid induction of membrane potential changes by extracellular nucleotides may be included by both eADP and eATP which induce large membrane depolarization changes in root hairs of Arabidopsis thaliana within seconds after the application. However, the application of phosphate has no effect, therefore negating the explanation that the depolarization was the result of phosphate released by hydrolysis of the applied nucleotides. Additionally, the application of ATP, GTP and ADP all induced large depolarizations; in contrast AMP, TTP and CTP did not. Dose-response studies revealed that half-maximal depolarization happened at about 0.4 mM for ATP, but at only about 10 μM for ADP, indicating that eADP was the more effective inducer of this response.

In animals nucleotide binding induces increased [Ca²⁺]_{cyt}, whether the receptor is either a P2X or P2Y type, however, no change in [Ca²⁺]_{cyt} induced by either eATP or eADP was observed when using dextran-conjugated calcium green as the reporter of Δ[Ca²⁺]_{cyt}. Additionally, an increase in [Ca²⁺]_{cyt} would be expected to decrease cytoplasmic streaming in the root hairs, but applied nucleotides had no effect on this, either. Additionally, eADP, but not eATP, induced a slight (e.g., about 22 to about 38%) increase in root hair growth.

A concentration of nucleotides in the ECM of plant cells of about 10 μM could be a higher concentration than would be actually found in the ECM of any plant cell, therefore one possible source that would expose root hairs to high concentrations of eATP would be root wounding, which would release cytoplasmic ATP to the outside of the cell. Several studies have measured cytoplasmic ATP concentrations at between about 1 and about 2 mM. Although, ATP released from root cells by wounding would be rapidly hydrolyzed by wall localized apyrases and phosphatases, it could be expected to reach and remain above 10 μM long enough to induce membrane depolarization changes.

If extracellular nucleotides were activating receptors in root hair cells, their failure to induce changes in the [Ca²⁺]_{cyt} of these cells indicate that the signalling pathways they induced were different from those induced by purinoceptors in animals. Alternatively, it was possible that the experimental set-up used by Lew and Dearnaley was not sensitive enough to detect the changes in [Ca²⁺]_{cyt} induced by eATP and eADP. [6] tested this possibility by using a very different methodology to assess the effects of extracellular nucleotides on [Ca²⁺]_{cyt}, e.g., the use of transgenic Arabidopsis plants constitutively expressing apoaequorin (Knight et al., Plant Cell 8: 489-503, 1996). When these plants take up the luminophore coelenterazine, the apoaequorin they are expressing is converted to the bioluminescent calcium sensor aequorin, which can then sensitively report changes in [Ca²⁺]_{cyt} by giving off light.

For example, Demidchik et al. [6] applied nucleotides to excised roots bathed in a buffered solution containing 10 mM CaCl₂ and found that as little as 300 nM ATP could induce a 2-fold increase in [Ca²⁺]_{cyt} in less than about 10 seconds. The non-hydrolyzable 2 meATP was almost as effective as ATP, indicating that agonist hydrolysis was not required for the response. Neither AMP or phosphate induced any significant change in [Ca²⁺]_{cyt}, and the pyrimidine UTP was ineffective below 100 μM, demonstrating the specificity of the nucleotide action. The P2-receptor antagonist pyridoxalphosphate-6-azophenyl-2′,4′-disulfonic acid (PPADS) and the calcium channel blocker gadolinium, both of which inhibit eATP action at some P2-receptors in animal cells, completely suppressed the response of roots to eATP. Taken together, the dose-responsiveness, substrate specificity and pharmacological profile suggested that the response was mediated by a distinct cell surface receptor, possibly an ion channel. The magnitude of the increase in [Ca²⁺]_{cyt}, from ca. 100 nM to over 400 nM after treatment with 1 μM ATP, was certainly sufficient to turn on calcium-dependent signaling pathways in plants.

Differences between the results can be attributed mainly to the different methodologies used (e.g., intact vs. excised roots; root hair response vs. whole root response; calcium green vs. aequorin reporter) [6 and 9]. Jeter et al. (2004), using the same type of aequorin-expressing Arabidopsis plants employed by Demidchik et al. (2003), showed that applied nucleotides could induce significant increases in [Ca²⁺]_{cyt} in intact Arabidopsis seedlings, with most of the luminescent signal coming from the aerial parts of the plant. They used similar controls as Demidchik et al. (2003), including all of the naturally occurring ATP derivatives such as AMP and phosphate, but using different poorly-hydrolysable nucleotide P2-receptor agonists (ATPγS, ADPβS, and AMPS), demonstrating other plant tissues besides the root can also respond to exogenously applied nucleotide derivatives, and confirming the specificity of the nucleotide effects. Further studies with calcium flux inhibitors, especially the use of the calcium chelator 1,2-bis(2-aminophenoxy)ethane-N,N,N′,N′-tetraacetic acid (BAPTA), strongly argue for an influx of calcium from the ECM, and, together with previous reports, indicate the presence of an plasma membrane-localized ion channel mediating the increased [Ca²⁺]cyt. Differences (e.g., higher threshold for induction observed in the seedlings) may have been due to intrinsic differences in the responsiveness of the two tissues, or to differences in the protocols employed (e.g., excised vs. intact tissue; 10 mM Ca²⁺ in measuring medium for roots vs. less than 100 μM Ca²⁺ in measuring medium for seedlings).

eATP treatments induced downstream gene expression changes known to influence hormone and stress responses, thus linking the initial [Ca²⁺]_{cyt} changes and later genetic changes that could mediate the growth and development of responding plants [10]. The application of 500 μM ATP or ADP, but not that of AMP or buffer, induced an increased abundance of mRNAs encoding various MAP kinases (e.g., ATMEKK1, ATMPK3, ATPK19), and the ethylene-related ERF2, ERF3, ERF4 and ACS6 genes. These gene expression changes were partially blocked in cells pre-treated with Gd³⁺ or a calcium chelator, revealing their dependence on an increase in [Ca²⁺]_{cyt}. The same genes had been shown to be up-regulated by touch and osmotic stresses [11], mechanical stimuli known to induce animal cells to release ATP into the ECM [12].

As assayed by the sensitive luciferin-luciferase method, stress stimuli do indeed induce measurable ATP release from young seedlings [10]. The touch stimulus was applied by gently shaking the seedlings, and the hypertonic stress was applied by briefly submerging the seedlings in 300 mM NaCl. Seedlings recovered from the stresses applied and appeared normal within 24 hours, indicating the released ATP did not come from irreversibly damaged cells.

Pathogen attack is another form of stress, and plants typically respond by the release of oligogalacturonides (OGA) in their ECM, that serves as a signalling molecule to induce defence responses through a transduction pathway that includes increase in [Ca²⁺]_{cyt} as an early step, e.g., OGA and ATPγS mutually enhanced each other's effects on the Δ[Ca²⁺]_{cyt}[10]. ADPβS with OGA together similarly increased [Ca²⁺]_{cyt}, whereas AMPS had a significant inhibitory effect. These results suggested that nucleotide derivatives could function together with OGA to modulate plant responses to wounding or herbivory, but they raise the question of the relationship between ATP and OGA signalling pathways.

Cessna and Low (2001) concluded that OGA induces increased [Ca²⁺]_{cyt} by Ca²⁺ release from internal stores, whereas other results showed that ATP induces increased [Ca²⁺]_{cyt} primarily by promoting Ca²⁺ uptake from the ECM [6 and 10]. Thus pathogen attack lead to the release of at least two different signalling agents, ATP and OGA, that act by different pathways to reinforce each other's stimulation of an early defence response, increased [Ca²⁺]_{cyt}, that is critical for downstream defence activities of the plant.

ATP involvement in defence responses is supported further by studies [13-14] that investigated whether the level of ATP that accumulates in the apoplastic space of a wound site is sufficient to induce superoxide production and downstream wound signaling responses. Micropipettes are used to collect multiple samples (e.g., several hundred fL each) of fluid accumulating at wound sites of Arabidopsis leaves and measured the [ATP] in these samples using the luciferin-luciferase method. The concentration was consistently in the 30 to 50 μM range.

For example, ATP samples as low as 500 nM applied into the intercellular spaces of Arabidopsis leaves was sufficient to rapidly induce significant superoxide production, as measured by the colorimetric method [15], which uses nitroblue tetrazolium as the staining agent. [13-14]. Delivery of equivalent concentrations of phosphate buffer or of AMP into the leaves had no significant effect on this response. That the response required the participation of NADPH oxidase, a key enzyme that catalyzes superoxide production in plants [16] and in animals [17], was demonstrated by the authors' observation that mutants disrupted in two genes that encode subunits of an NADPH oxidase homolog also do not accumulate significant superoxide in response to eATP [13-14].

Various inhibitor treatments provided insight on the signaling pathway leading from ATP to superoxide production. Inhibitors of receptors that initiate eATP responses in animals, such as PPADS, were able to block the response. Cation channel blockers, calcium chelators, and calmodulin antagonists also blocked this ATP response, implicating increases in [Ca²⁺]_{cyt} and the activation of calmodulin as intermediate signaling steps. Perhaps most importantly, pre-treatment of leaves with relatively low concentrations of potato apyrase (i.e., an enzyme that efficiently hydrolyzes ATP) significantly reduced the level of superoxide induced by wounding, arguing that it is likely ATP itself, and not a breakdown product, eliciting the response.

Genes that are induced by various stresses, including genes involved in the biosynthesis of jasmonates and ethylene, LOX2 and ACS6, respectively, were up-regulated by eATP at the same micromolar concentrations that induced superoxide production, further supporting a role for eATP as a signal. Message abundance for PAL1, which is a well-studied stress-induced and ROS-induced gene, was also increased by eATP, and this effect was blocked by P2 receptor antagonists [13-14]. Taken together these results indicate that the release of ATP at wound sites can serve as an early signal to induce superoxide production and downstream gene expression changes typically induced by the wound stimulus.

Slower Growth Response Changes Induced by eATP. Superoxide production can have growth regulatory effects that can be both promotive and inhibitory of growth [17-18], Tang and colleagues [19-20] demonstrated that relatively high concentrations of applied ATP and ADP (3 mM) and lower concentrations of the relatively non-hydrolyzable nucleotides ATPγS and ADPβS (0.3 mM) could inhibit the straight growth of roots, and that concentrations of these nucleotides about three times lower could inhibit gravitropic growth of roots without significantly inhibiting their straight growth.

Because of the strong relationship of auxin transport to growth, [20] tested whether the growth inhibition induced by applied ATP could be mediated by the eATP effects on auxin transport. Their results indicated that both in maize and Arabidopsis roots the same concentrations of ATP that inhibited gravitropic growth also inhibited auxin transport.

To investigate by what mechanism ATP was having its effects on growth, [20] carried out a variety of controls to render unlikely some trivial explanations, such as that the effects were due to ATP-induced pH changes or chelation of divalent cations or to phosphate released from the hydrolysis of the applied nucleotides. Two possible mechanisms of ATP action include: (1) eATP could reduce the steepness of the ATP gradient across the plasma membrane and thus inhibit the transport effectiveness of an MDR transporter that has been implicated in auxin transport [21]; and (2) the applied ATP could be acting through the more traditional mechanism of activating a P2-purinoceptors.

If mM concentrations of eATP are needed to activate some plant receptors, then these receptors would have to be far less sensitive than the mammalian ones, or only a small fraction of the applied ATP and ADP would be reaching the receptor site, with the rest being rapidly hydrolyzed or otherwise altered. In the animal literature, P2X purinoceptors were originally thought to be quite insensitive, responding only to mM levels of ATP, but now it appears that P2X receptors can respond to nM levels of ATP, but these same levels desensitize the receptors so that they subsequently will respond only to much higher (mM) doses (Rettinger and Schmalzing 2004). The same desensitization phenomenon could significantly raise the threshold of plant responsiveness to external nucleotides.

Schopfer [22] has presented data favoring a promotive role for superoxide in plant growth. Tang [19] showed that concentrations of ATP in the range of 100 to 200 μM could significantly promote hypocotyl growth in etiolated seedlings of Arabidopsis and even lower doses of ATPγS and ADPβS, e.g., in the 10 to 20 μM range, can stimulate hypocotyl growth, while doses above 400 μM inhibit growth. Comparing these results reveals that the threshold for inhibiting growth is about 10 times lower when the poorly-hydrolysable nucleotides are applied [20]. Taken together the data show that nucleotide hydrolysis is not required for its growth effects and suggest that the less sensitive responses to ATP and ADP may be due to the rapidity with which these nucleotides are hydrolyzed in the plant cell wall, where acid and alkaline phosphatases abound.

To relate the above results to physiology, Coco et al. [23] have proposed that sites of active delivery of secretory vesicles are sites of release of high concentrations of ATP, because secretory vesicles contain concentrations of ATP near mM (Pugielli et al. 1999), and they would unload the ATP into the ECM when they fuse with the plasma membrane. In plants, cell growth would be accompanied by the delivery of ATP to plant cell walls, because sites of active growth in plants are also sites of active delivery of secretory vesicles [24].

Since high levels of ATP can inhibit growth, one could also postulate that control of [eATP] at growth sites would be a mechanism of growth control. For example, treatment of wild-type pollen with either μM levels of ATPγS or with inhibitors of the ATP-hydrolyzing enzyme apyrase inhibits pollen germination [25]. Knocking out AtApy1 and AtApy2, two closely related apyrases that are both strongly expressed in wild-type pollen, also blocks pollen germination [25]. By sequence analysis there appear to be seven different apyrases in Arabidopsis, and it is important to determine which of these function as ectoapyrases and thus as potential regulators of extracellular nucleotide agents that may serve as growth regulators in plants.

Legume apyrases have been strongly implicated in the process of Nod signaling [26], and appear to play a role in plant defense against pathogens [27]. To the extent that members of this family control [eATP] they could obviously play major roles in growth control. Progress in defining the role(s) of apyrases in nodulation will be synergistic with progress in defining the role(s) of apyrases in growth control in Arabidopsis and other non-nodulating species. Quite likely, the apyrase studies in legumes may also catalyze more rapid progress in defining the role(s) of extracellular nucleotides in plant growth and development.

Extracellular signaling functions for adenosine nucleoside tri- and diphosphates have been well documented in animals for several decades. Early observations of nucleotide effects on plant biological processes suggest that extracellular ATP could be a signaling agent in plants as well. For example, these reports show that exogenous application of ATP increases lily pollen tube mitotic activity [5] modulates stomatal guard cell aperture [4], and promotes the closure of the Venus fly trap [2].

Externally applied caffeine, another purine derivative, induces pronounced inhibitory effects on plant developmental and cellular processes, including sieve plate formation [28], tracheary element differentiation [29] and pollen tube growth [30]. Importantly, cytokinins, which are adenine derivatives, are among the major classes of plant hormones regulating plant growth and development [31].

Although cytokinin has been accepted as a bona fide extracellular signaling agent in plants, further evidence of a cell-cell signaling function for extracellular ATP has been required to circumvent arguments that its effects are simply due to alterations in the cells' energy status. The presence of prospective ATP release mechanisms has supported the notion that ATP is present in the plant extracellular matrix (ECM), thereby satisfying the requirement for an endogenous signal source. Cell lysis is a passive means by which cytoplasmic ATP can exit any cell, and plant cells are undoubtedly subject to this simplistic mechanism for ATP release during events such as wounding or herbivory.

Exocytosis of secretory vesicles containing ATP [32], as well as efflux through anion channels [33,34] or in association with Multidrug Resistance (MDR) Transporters [35] all represent potential ATP efflux mechanisms characterized in animals, and similar mechanisms are possible in plants as well. In this regard, expression of at least one plant MDR transporter homologue, AtPGP1 (Arabidopsis thaliana p-glycoprotein) in yeast has been shown to increase ATP release into the growth medium, and to similarly increase extracellular ATP concentrations when overexpressed in transgenic Arabidopsis [36].

The conservation of key regulatory players further substantiates a putative signaling function for extracellular ATP in the plant ECM. ATP/ADP hydrolytic activity could participate in the termination of an ATP signal, the maintenance of responsiveness to such a signal, and in the recycling of the ATP constituent components. For example, numerous apyrase (NTPDase) homologues, at least some of which have been shown to have an extracellular localization, have been cloned from a wide variety of plants including Solanum tuberosum [37], Pisum sativum [38], Dolichos biflorus [39] and Arabidopsis thaliana [40], among others [41,42]. The NTP/NDP hydrolyzing activity of these enzymes, presumably along with the action of nucleotidases and phosphatases, has been proposed to participate in phosphate scavenging in plants [43] and the maintenance of xenobiotic resistance in association with MDR transporters [36]. Of particular interest, apyrases have been implicated in a variety of developmental processes as well, including pollen germination [44], nodulation [45, 46] and growth (Roux et al., 2006).

The present invention provides a method of modulation of drug resistance in plants, particularly herbicide resistance, through the addition of extracellular nucleotides. The addition of extracellular nucleotides provides a cascade that ultimately results in changes in the expression of specific genes (e.g., MDR-ABC transporter and an ecto-phosphatase) that in turn results in resistance to certain plant hormones, drugs, fungicides and herbicides. In addition, the extracellular nucleotides may result in post translational modifications may include phosphorylation, adenylation, glycosylation, ubiquitinylation, acetylation, methylation, farnesylation, myristilation and sulfation.

In addition, the present invention modulates the effect of herbicides on plants. The skilled artisan will recognize that a vast array of hormones, herbicides that resemble drugs and chemicals may be affected by the extracellular nucleotides of the present invention. Nonlimiting examples of plant hormones include cytokinin, auxins, gibberellins and brassinosteroids. A nonlimiting list of chemicals includes chloro-N-(ethoxymethyl)-N-(2-ethyl-6-methyl-phenyl)acetamide; 5-[2-chloro-4-(trifluoromethyl)-phenoxy]-2-nitro-benzoic acid; 2-propenal; 2-chloro-N-(2,6-diethylphenyl)-N-(methoxy-methyl)acetamide; 2-propen-1-ol; N-ethyl-N′-(1-methylethyl)-6-(methyl-thio)-1,3,5-triazine-2,4-diamine; 1H-1,2,4-triazol-3-amine; ammonium sulfamate; arsenic acid; methyl[(4-aminophenyl)sulfonyl]carbamate; N-ethyl-6-methoxy-N′-(1-methylethyl)-1,3,5-tri-azine-2,4-diamineatrazine; 6-chloro-N-ethyl-N′-(1-methylethyl)-1,3,5-tri-azine-2,4-diamine; 2-[2,4-dichloro-5-(2-propynyloxy)phenyl]-5,6,7,8-tetrahydro-1,2,4-tri-azolo[4,3-a]pyridin-3(2H)-one; N-[[(4,6-dimethoxy-2-pyrimidinyl)amino]carbonyl]-1-methyl-4-(2-methyl-2H-tetrazol-5-yl)-1H-pyrazole-5-sulfonamide; 4-chloro-2-butynyl 3-chlorophenylcarbamate; 1-methylpropyl 3-chlorophenylcarbamate; 4-chloro-2-oxo-3(2H)-benzothiazoleacetic acid; N-butyl-N-ethyl-2,6-dinitro-4-(tri-fluoromethyl)benzenamine; 2-[[[[[(4,6-dimethoxy-2-pyri-midinyl)amino]carbonyl]amino]sulfonyl]methyl]benzoic acid; O,O-bis(1-methylethyl)S-[2-[(phenyl-sulfonyl)amino]ethyl]phosphorodithioate; 3-(1-methylethyl)-(1H)-2,1,3-benzo-thiadiazin-4(3H)-one 2,2-dioxide; [(benzoylamino)oxy]acetic acid; 3,5-dimethyl-N-(1-methylethyl)-N-(phenyl-methyl)benzamide; N-[4-(ethylthio)-2-(trifluoro-methyl)phenyl]methanesulfonamide; N-benzoyl-N-(3,4-dichlorophenyl)-DL-alanine; N-2-benzothiazolyl-N′-methylurea; methyl 5-(2,4-dichlorophenoxy)-2-nitrobenzoate; borax; sodium tetraborate; 5-bromo-6-methyl-3-(1-methyl-propyl)-2,4(1H,3H)pyrimidinedione; bromofenoxim; 3,5-dibromo-4-hydroxybenzaldehyde O-(2,4-dinitrophenyl)oxime; 3,5-dibromo-4-hydroxybenzonitrile; N-(butoxymethyl)-2-chloro-N-(2,6-diethyl-phenyl)acetamide; 2,2-dimethyl-N-(1-methylethyl)-N-phenyl-methyl)propanamide; O-ethyl O-(5-methyl-2-nitrophenyl) 1-methylpropylphosphoramidothioate; 3-[5-(1,1-dimethylethyl)-1,3,4-thiadiazol-2-yl]-4-hydroxy-1-methyl-2-imidazolidinone; 4-(1,1-dimethylethyl)-N-(1-methyl-propyl)-2,6-dinitrobenzenamine; N′-(4-chlorophenyl)-N-methyl-N-(1-methyl-2-propynyl)urea; S-ethyl-bis(2-methylpropyl)carbamothioate cacodylic acid dimethyl arsinic acid; (phenylimino)di-2,1-ethanediyl bis(3,6-dichloro-2-methoxybenzoate); N-ethyl-2-[[(phenylamino)carbonyl]oxy]propanamide; 2-chloro-N,N-di-2-propenylacetamide; 2-dichloro-5-[4-(difluoromethyl)-4,5-dihydro-3-methyl-5-oxo-1H-1,2,4-triazol-1-yl]-4-fluorobenzenepropanoic acid; 2-chloro-N,N-diethylacetamide; 2-chloro-2-propenyl-diethylcarbamodithioate; 2-chloroethyl(3-chlorophenyl)carbamate; 3-amino-2,5-dichlorobenzoic acid; 6-chloro-N,N,N′,N′-tetraethyl-1,3,5-triazine-2,4-diamine; N′-(4-bromo-3-chlorophenyl)-N-methoxy-N-methylurea; 1-methyl-2-propynyl (3-chlorophenyl)carbamate; 2-chloro-9-hydroxy-9H-fluorene-9-carboxylic acid; 2-[[[[(4-chloro-6-methoxy-2-pyrimidinyl)amino]carbonyl]amino]sulfonyl]benzoic acid; N′-[4-(4-chlorophenoxy)phenyl]-N,N-dimethylurea; 1-methylethyl-3-chlorophenylcarbamate; 2-chloro-N-[[(4-methoxy-6-methyl-1,3,5-triazin-2-yl)amino]carbonyl]benzenesulfonamide; 2,6-dichlorobenzenecarbothiamide; N′-(3-chloro-4-methylphenyl)-N,N-dimethylurea; exo-1-methyl-4-(1-methylethyl)-2-[(2-methyl-phenyl)methoxy]-7-oxabicyclo[2.2.1]heptane; cis-2,5-dimethyl-N-phenyl-1-pyrrolidinecarboxamide; clethodim (E,E)-2-[1-[[(3-chloro-2-propenyl)oxy]imino]propyl]-5-[2-(ethylthio)propyl]-3-hydroxy-2-cyclohexen-1-one; 2-[4-(4-chlorophenoxy)phenoxy]propanoic acid; 2-[(2-chlorophenyl)methyl]-4,4-dimethyl-3-isoxazolidinone; (E,E)-2-[1-[[(3-chloro-2-propenyl)oxy]imino]butyl]-5-[2-(ethylthio)propyl]-3-hydroxy-2-cyclohexen-1-one; 3-chloro-2-[[(5-ethoxy-7-fluoro[1,2,4]triazolo[1,5-c]pyrimidin-2yl)sulfonyl]amino]benzoic acid; 3,6-dichloro-2-pyridinecarboxylic acid; copper sulfate; (4-chlorophenoxy)acetic acid; 4-(4-chlorophenoxy)butyric acid; 1-chloro-N′-(3,4-dichlorophenyl)-N—N-dimethylformamidine; 2-(4-chlorophenoxy)propionic acid; 2-chloro-1-methylethyl (3-chlorophenyl)carbamate; 2-[[4-chloro-6-(ethylamino)-1,3,5-triazin-2-yl]amino]-2-methylpropanenitrile; S-ethyl cyclohexylethylcarbamothioate; N-[[[2-(cyclopropylcarbonyl)phenyl]amino]sulfonyl]-N′-(4,6-dimethoxy-2-pyrimidinyl)urea; N′-cyclooctyl-N,N-dimethylurea; (R)-2-[4-(4-cyano-2-fluoro-phenoxy)phenoxy]propanoic acid; 1-methyl-4-phenylpyridinium; 6-chloro-N-cyclopropyl-N′-(1-methyl-ethyl)-1,3,5-triazine-2,4-diamine; N-[5-(2-chloro-1,1-dimethylethyl)-1,3,4-thiadiazol-2-yl]cyclopropanecarboxamide; N-(3,4-dichlorophenyl)cyclopropanecarboxamide; 2,4-D (2,4-dichlorophenoxy)acetic acid; 3,4-DA (3,4-dichlorophenoxy)acetic acid; 2,2-dichloropropanoic acid; tetrahydro-3,5-dimethyl-2H-1,3,5-thiadiazine-2-thione; 2,4-DB 4-(2,4-dichlorophenoxy)butanoic acid; 3,4-DB 4-(3,4-dichlorophenoxy)butanoic acid; 1,2-dichlorobenzene; dimethyl 2,3,5,6-tetrachloro-1,4-benzene-dicarboxylate; N,N′-bis(2,2,2-trichloro-1-hydroxyethyl)urea; 2,4-DEB 2-(2,4-dichlorophenoxy)ethyl benzoate; 2-chloro-N-(2,6-dimethylphenyl)-N-[(2-methyl-propoxy)methyl]acetamide; tris[2-(2,4-dichlorophenoxy)ethyl]phosphate; ethyl[3-[[(phenylamino)carbonyl]oxy]phenyl]carbamate; N-methyl-N′-(1-methylethyl)-6-(methyl-thio)-1,3,5-triazine-2,4-diamine; S-(2,3-dichloro-2-propenyl) bis(1-methyl-ethyl)carbamothioate; 3,6-dichloro-2-methoxybenzoic acid; 2,6-dichlorobenzonitrile; 3,4-dichlorobenzenemethanol; 2-(2,4-dichlorophenoxy)propanoic acid; 2-[4-(2,4-dichlorophenoxy)phenoxy]propanoic acid; N-(3,4-dichlorophenyl)-2-methyl-2-propenamide; N-(chloroacetyl)-N-(2,6-diethylphenyl)glycine; N-(2,6-dichlorophenyl)-5-ethoxy-7-fluoro[1,2,4]triazolo[1,5-c]pyrimidine-2-sulfonamide; (E)-4-[4-[4-(trifluoromethyl)phenoxy]phenoxy]-2-pentenoic acid; N′-[4-(4-methoxyphenoxy)phenyl]-N,N-dimethylurea; 1,2-dimethyl-3,5-diphenyl-1H-pyrazolium; 2-chloro-N-(2,6-dimethylphenyl)-N-(2-methoxy-ethyl)acetamide; N-(1,2-dimethylpropyl)-N′-ethyl-6-(methylthio)-1,3,5-triazine-2,4-diamine; N3,N3-diethyl-2,4-dinitro-6-(trifluoro-methyl)-1,3-benzenediamine; 2-(1-methylbutyl)-4,6-dinitrophenol; 2-(1-methlpropyl)-4,6-dinitrophenol; 2-(1,1-dimethylethyl)-4,6-dinitrophenol; N,N-dimethyl-a-phenyl benzeneacetamide; 6-(ethylthio)-N,N′-bis(1-methyl-ethyl)-1,3,5-triazine-2,4-diamine; 6,7-dihydrodipyrido[1,2-a:2′,1′-c[pyra-zinediium ion dithiopyr S,S-dimethyl 2-(difluoromethyl)-4-(2-methyl-propyl)-6-(trifluoromethyl)-3,5-pyridine-dicarbothioate; N′-(3,4-dichlorophenyl)-N,N-dimethylurea; 2-methyl-4,6-dinitrophenol 3,4-DP 2-(3,4-dichlorophenoxy)propanoic acid; disodium salt of MAA; ethyl bis(2-ethylhexyl)phosphinate; N-(4-chloro-6-ethylamino-1,3,5-tri-azin-2-yl)glycine; 7-oxabicyclo[2.2.1]heptane-2,3-dicarboxylic acid; Sodium salt of endothal; S-ethyl dipropyl carbamothioate; 2-(2,4,5-trichlorophenoxy)ethyl-2,2-dichloropropanoate; N-ethyl-N-(2-methyl-2-propenyl)-2,6-dinitro-4-(trifluoromethyl)benzenamine; thametsulfuron-2-[[[[[[4-ethoxy-6-(methylamino)-1,3,5-triazin-2-yl]amino]carbonyl]amino]sulfonyl]benzoic acid; N-(5-ethylsulfonyl-1,3,4-thiadiazol-2-yl)-N,N′-dimethylurea; S-ethyl diethylcarbamothioate ethofumesate-2-ethoxy-2,3-dihydro-3,3-dimethyl-5-benzo-furanyl methanesulfonate; diethyl thioperoxydicarbonate; 2,3,6-trichlorobenzeneacetic acid; 2-[4-[(6-chloro-2-benzoxazolyl)oxy]phenoxy]propanoic acid; N,N-dimethyl-N′-phenylurea; TCA salt of fenuron; N-benzoyl-N-(3-chloro-4-fluoro-phenyl)-DL-alanine; 2-[4-[[5-(trifluoromethyl)-2-pyridinyl]oxy]phenoxy]propanoic acid; (R)-2-[4-[[5-(trifluoromethyl)-2-pyridinyl]oxy]phenoxy]propanoic acid; N-(2-chloroethyl)-2,6-dinitro-N-propyl-4-(trifluoromethyl)benzenamine; N-(2,6-difluorophenyl)-5-methyl[1,2,4]triazolo[1,5-a]pyrimidine-2-sulfonamide; [2-chloro-4-fluoro-5(1,3,4,5,6,7-hexa-hydro-1,3-dioxo-2H-isoindol-2-yl)phenoxy]acetic acid; 2-[7-fluoro-3,4-dihydro-3-oxo-4-(2-propynyl)-2H-1,4-benzoxazin-6-yl]-4,5,6,7-tetrahydro-1H-isoindole-1,3(2H)-dione; N,N-dimethyl-N′-[3-(trifluoromethyl)phenyl]urea; 3-chloro-4-(chloromethyl)-1-[3-(trifluoro-methyl)phenyl]-2-pyrrolidinone; 2-nitro-1-(4-nitrophenoxy)-4-trifluoro-methylbenzene; carboxymethyl 5-[2-chloro-4-(trifluoromethyl)phenoxy]-2-nitrobenzoate; 1-methylethyl 2-chloro-5-[3,6-dihydro-3-methyl-2,6-dioxo-4-(trifluoromethyl)-1(2H)-pyrimidinyl]benzoate; 2-[[[[(4,6-dimethoxy-2-pyrimidinyl)amino]carbonyl]amino]sulfonyl]-6-(trifluoromethyl)-3-pyridinecarboxylic acid; 1-methyl-3-phenyl-5-[3-(trifluoromethyl)phenyl]-4(1H)-pyridinone; [(4-amino-3,5-dichloro-6-fluoro-2-pyri-dinyl)oxy]acetic acid; 5(methylamino)2-phenyl-4-[3-(trifluoro-methyl)phenyl]-3(2H)-furanone; 5-[2-chloro-4-(trifluoromethyl)phenoxy]-N-(methylsulfonyl)-2-nitrobenzamide; ethyl hydrogen(aminocarbonyl)phosphonate glufosinate 2-amino-4-(hydroxymethylphosphinyl)butanoic acid; N-(phosphonomethyl)glycine; 5-[2-chloro-6-fluoro-4-(trifluoromethyl)phenoxy]-N-(ethylsulfonyl)-2-nitrobenzamide; 2-[4-[[3-chloro-5-(trifluoromethyl)-2-pyridinyl]oxy]phenoxy]propanoic acid; potassium hexafluoroarsenate; 3-cyclohexyl-6(dimethylamino)-1-methyl-1,3,5-triazine-2,4(1H,3H)-dione; 2-[4,5-dihydro-4-methyl-4-(1-methylethyl)-5-oxo-1H-imidazol-2-yl]-4(and 5)-methyl-benzoic acid; 2-[4,5-dihydro-4-methyl-4-(1-methyl-ethyl)-5-oxo-1H-imidazol-2-yl]-5-(meth-oxymethyl)-3-pyridinecarboxylic acid; 2-[4,5-dihydro-4-methyl-4-(1-methyl-ethyl)-5-oxo-1H-imidazol-2-yl]-3-pyridinecarboxylic acid; 2-[4,5-dihydro-4-methyl-4-(1-methyl-ethyl)-5-oxo-1H-imidazol-2-yl]-3-quinolinecarboxylic acid; 2-[4,5-dihydro-4-methyl-4-(1-methyl-ethyl)-5-oxo-1H-imidazol-2-yl]-5-ethyl-3-pyridinecarboxylic acid; 4-hydroxy-3,5-diiodobenzonitrile; 6-chloro-N,N-diethyl-N′-(1-methyl-ethyl)-1,3,5-triazine-2,4-diamine; O-(1-methylethyl)carbonodithioate; N-(2-methylpropyl)-2-oxo-1-imidazolidine; carboxamide; 5-bromo-6-methyl-3-(1-methyl-ethyl)-2,4(1H,3H)-pyrimidinedione; 6-(1,1-dimethylethyl)-4-[(2-methylpropylidene)amino]-3-(methylthio)-1,2,4-triazin-5-(4H)-one; 4-(1-methylethyl)-2,6-dinitro-N,N-dipropylbenzenamine; N,N-dimethyl-N′-[4-(1-methylethyl)phenyl]urea; N′-[5-(1,1-dimethylethyl)-3-isoxazolyl]-N,N-dimethylurea; N-[3-(1-ethyl-1-methylpropyl)-5-isox-azolyl]-2,6-dimethoxybenzamide; 3-[[(dimethylamino)carbonyl]amino]phenyl(1,1-dimethylethyl)carbamate; KOCN potassium cyanate; 2-ethoxy-1-methyl-2-oxoethyl 5-[2-chloro-4-(trifluoromethyl)phenoxy]-2-nitrobenzoate; 3-cyclohexyl-6,7-dihydro-1H-cyclopenta-pyrimidine-2,4(3H,5H)-dione; N′-(3,4-dichlorophenyl)-N-methoxy-N-methylurea MAA methylarsonic acid; monoammonium salt of MAA; maleic hydrazide; 1,2-dihydro-3,6-pyridazinedione; (4-chloro-2-methylphenoxy)acetic acid; 4-(4-chloro-2-methylphenoxy)butanoic acid; 2-(4-chloro-2-methylphenoxy)propanoic acid; N-[2,4-dimethyl-5-[[(trifluoromethyl)sulfonyl]amino]phenyl]acetamide; Sodium salt of metham; 4-amino-3-methyl-6-phenyl-1,2,4-trizin-5(4H)-one; N-(2-methyl-2-propenyl)-2,6-dinitro-N-propyl-4-(trifluoromethyl)benzenamine; methylcarbamodithioic acid; 2-(3,4-dichlorophenyl)-4-methyl-1,2,4-oxadiazolidine-3,5-dione; N-(2-benzothiazolyl-N,N′-dimethylurea N-(3-methoxypropyl)-N′-(1-methylethyl)-6-(methylthio)-methoprotryn 1,3,5-triazine-2,4-diamine; methyl bromide; bromomethane; N′-(4-bromophenyl)-N-methoxy-N-methylurea; (2-methoxy-1-methylethyl)acetamide; 2-chloro-N-(2-ethyl-6-methylphenyl)-N-metosulam; N-(2,6-dichloro-3-methylphenyl)-5,7-dimethoxy[1,2,4]triazolo[1,5-a]pyrimidine-2-sulfonamide; N′-(3-chloro-4-methoxyphenyl)-N,N-dimethylurea; 4-amino-6-(1,1-dimethylethyl)-3-(methylthio)-1,2,4-triazin-5(4H)-one; 2-[[[[(4-methoxy-6-methyl-1,3,5-triazin-2-yl)amino]carbonyl]amino]sulfonyl]benzoic acid; S-ethyl hexahydro-1H-azepine-1-carbothioate; N-(4-chlorophenyl)-2,2-dimethylpentanamide; N′-(4-chlorophenyl)-N-methoxy-N-methylurea; N′-(4-chlorophenyl)-N,N-dimethylurea; monuron; N,N-diethyl-2-(1-naphthalenyloxy)propanamide; 2-[(1-naphthalenylamino)carbonyl]benzoic acid; N-butyl-N′-(3,4-dichlorophenyl)-N-methylurea; 2-[[[[(4,6-dimethoxy-2-pyrimidinyl)amino]carbonyl]amino]sulfonyl]-N,N-dimethyl-3-pyridinecarboxamide; 4-(methylsulfonyl)-2,6-dinitro-N,N-dipropylbenzenamine; 2,4-dichloro-1-(4-nitrophenoxy)benzene; 2-chloro-1-(4-nitrophenoxy)-4-(trifluoromethyl)benzene; N,N-dimethyl-N′-(octahydro-4,7-methano-1H-inden-5-yl)urea; 4-chloro-5-(methylamino)-2-(3-(trifluoromethyl)phenyl)-3(2H)-pyridazinone; 2,3,4,4,5,5,6,6-octachloro-2-cyclohexen-1-one oryzalin 4-(dipropylamino)-3,5-dinitrobenzenesulfonamide; 3-[2,4-dichloro-5-(1-methylethoxy)phenyl]-5-(1,1-dimethylethyl)-1,3,4-oxadiazol-2-(3H)-one; 2-chloro-1-(3-ethoxy-4-nitrophenoxy)-4-(trifluoromethyl)benzene; 1,1′-dimethyl-4,4′-bipyridinium ion; chlorinated benzoic acid; pentachlorophenol; S-propyl butylethylcarbamothioate; pelargonic acid; nonanoic acid; pendimethalin; N-(1-ethylpropyl)-3,4-dimethyl-2,6-dinitrobenzenamine; 1,1,1-trifluoro-N[2-methyl-4-phenylsulfonyl)phenyl]methanesulfonamide; 3-[[(1-methylethoxy)carbonyl]amino]phenyl ethylphenylcarbamate; 3-[(methoxycarbonyl)amino]phenyl(3-methyl-phenyl)carbamate; 4-amino-3,5,6-trichloro-2-pyridinecarboxylic acid; S-[2-(2-methyl-1-piperidinyl)-2-oxo-ethyl]O,O-dipropylphosphorodithioate; (acetato-O)phenylmercury potassium azide potassium azide; 2-[[[[[4,6-bis(difluoromethoxy)-2-pyrimidinyl]amino]carbonyl]amino]sulfonyl]benzoic acid; 2-[[4-chloro-6-(cyclopropylamino)-1,3,5-triazine-2-yl]amino]-2-methylpropanenitrile; 2,4 dinitro-N3,N3-dipropyl-6-(trifluoromethyl)-1,3-benzenediamine; N-(cyclopropylmethyl)-2,6-dinitro-N-propyl-4-(trifluoromethyl)benzenamine; N-[4-chloro-6-(1-methylethylamino)-1,3,5-triazine-2-yl]glycine; 6-methoxy-N,N′-bis(1-methylethyl)-1,3,5-triazine-2,4-diamine; N,N′-bis(1-methylethyl)-6-(methylthio)-1,3,5-triazine-2,4-diamine; 3,5-dichloro (N-1,1-dimethyl-2-propynyl)benzamide; 2-chloro-N-(1-methylethyl)-N-phenylacetamide; N-(3,4-dichlorophenyl)propanamide; (R)-2-[[(1-methylethylidene)amino]oxy]ethyl 2-[4-[(6-chloro-2-quinoxalinyl)oxy]phenoxy]propanoate; 6-chloro-N,N′-bis(1-methylethyl)-1,3,5-triazine-2,4-diamine; 1-methylethyl phenylcarbamate; N-[[4-(dipropylamino)-3,5-dinitrophenyl]sulfonyl]-S,S-dimethylsulfilimine; 2-chloro-N-(1-methyl-2-propynyl)-N-phenylacetamide; 5-amino-4-chloro-2-phenyl-3(2H)-pyridazinone; 2,3,5-trichloro-4-pyridinol; O-(6-chloro-3-phenyl-4-pyridazinyl) S-octyl carbonothioate; 2-chloro-6-[(4,6-dimethoxy-2-pyrimidinyl)thio]benzoic acid; 3,7-dichloro-8-quinolinecarboxlic acid; 2,2-dichloro-N-(3-chloro-1,4-dihydro-1,4-dioxo-2-naphthalenyl)acetamide; 2-[4-[(6-chloro-2-quinoxalinyl)oxy]phenoxy]propanoic acid; N-[[(4,6-dimethoxy-2-pyrimidinyl)amino]carbonyl]-3 (ethylsulfonyl)-2-pyridinesulfonamide; N-ethyl-6-methoxy-N′-(1-methylpropyl)-1,3,5-triazine-2,4-diamine; 2-[1-(ethoxyimino)butyl]-5-[2-(ethyl-thio)propyl]-3-hydroxy-2-cyclohexen-1-one; 2-(2,4-dichlorophenoxy)ethyl hydrogen sulfate; N-(2-methylcyclohexyl)-N′-phenylurea; 2-(2,4,5-trichlorophenoxy)propanoic acid; 6-chloro-N,N′-diethyl-1,3,5-triazine-2,4-diamine; N,N′-diethyl-6-methoxy-1,3,5-triazine-2,4-diamine; N,N′-diethyl-6-(methylthio)-1,3,5-triazine-2,4-diamine; sodium arsenite; sodium azide; sodium chlorate; N-(3-chloro-4-methylphenyl)-2-methyl-pentanamide; N-[2,4-dichloro-5-[4-(difluoromethyl)-4,5-diydro-3-methyl-5-oxo-1H-1,2,4-triazol-1-yl]phenyl]methanesulfonamide; 2-[[[[(4,6-dimethyl-2-pyrimidinyl)amino]carbonyl]amino]sulfonyl]benzoic acid; methyl(3,4-dichlorophenyl)carbamate; 2,4,5-T (2,4,5-trichlorophenoxy)acetic acid; 2,4,5-TB 4-(2,4,5-trichlorophenoxy)butanoic acid; 2,3,6-trichlorobenzoic acid; trichloroacetic acid; N-[5-(1,1-dimethylethyl)-1,3,4-thiadiazol-2-yl]-N,N′-dimethylurea; 5-chloro-3-(1,1-dimethylethyl)-6-methyl-2,4(1H,3H)-pyrimidinedione; N-(butoxymethyl)-2-chloro-N-[2-(1,1-dimethyl-ethyl)-6-methylphenyl]acetamide; N-(1,1-dimethylethyl)-N′-ethyl-6-methoxy-1,3,5-triazine-2,4-diamine; 6-chloro-N-(1,1-dimethylethyl)-N′-ethyl-1,3,5-triazine-2,4-diamine; 2,6-bis(1,1-dimethylethyl)-4-methyl-phenyl methylcarbamate; N-(1,1-dimethylethyl)-N′-ethyl-6-(methyl-thio)-1,3,5-triazine-2,4-diamine N,N-dimethyl-N′-[3-(1,1,2,2-tetra-fluoroethoxy)phenyl]urea; N,N′-dimethyl-N-[5-(trifluoromethyl)-1,3,4-thiadiazol-2-yl]urea; methyl-2-(difluoromethyl)-5-(4,5-dihydro-2-thi-azolyl)-4-(2-methylpropyl)-6-(trifluoromethyl)-3-pyridinecarboxylate; 3-[[[[(4-methoxy-6-methyl-1,3,5-triazin-2-yl)amino]carbonyl]amino]sulfonyl]-2-thiophenecarboxylic acid; S-[(4-chlorophenyl)methyl]diethylcarbamothioate; 2,2,3-trichloropropionic acid; S-(2,3,3-trichloro-2-propenyl) bis(1-methyl-ethyl)carbamothioate; 2-(2-chloroethoxy)-N-[[(4-methoxy-6-methyl-1,3,5-triazin-2-yl)amino]carbonyl]benzenesulfonamide; 2-[[[[(4-methoxy-6-methyl-1,3,5-triazin-2-yl)methylamino]carbonyl]amino]sulfonyl]benzoic acid; tricamba 2,3,5-trichloro-6-methoxy benzoic acid; triclopyr [(3,5,6-trichloro-2-pyridinyl)oxy]acetic acid; 2-(3,5-dichlorolphenyl)-2-(2,2,2-trichloro-ethyl)oxirane; 6-chloro-N,N,N′-triethyl-1,3,5-tri-azine-2,4-diamine; 2,6-dinitro-N,N-dipropyl-4-(trifluoro-methyl)benzenamine; 2-[[[[[4-(dimethylamino)-6-(2,2,2-trifluoro-ethoxy)-1,3,5-triazin-2-yl]amino]carbonyl]amino]sulfonyl]-3-methylbenzoic acid; methyl N′-(4-chlorophenyl)-N,N-dimethyl-carbamidate; 1-[(2,3,6-trichlorophenyl)methoxy]-2-propanol; S-propyl dipropylcarbamothioate; 2-chloro-N-(2,3-dimethylphenyl)-N-(1-methyl-ethyl)acetamide.

Fungicides include but not limited to picoxystrobin azoxystrobin; triadimenol; chlorothalonil; propiconazole; cyproconazole; metconazole; pyraclostrobin; fenpropimorph; quinoxyfen; fluoxastrobin; prothioconazole; fluquinconazole; metrafenone; tebuconazole; cyproconazole; quinoxyfen; flusilazole; bromuconazole; tetraconazole; fenbuconazole; kresoxim-methyl; epoxiconazole; flusilazole; kresoxim-methyl; epoxiconazole; fenpropimorph; spiroxamine; kresoxim-methyl; epoxiconazole; pyraclostrobin; epoxiconazole; difenoconazole; flutriafol; prochloraz; prothioconazole; fenbuconazole; flusilazole; prochloraz; fenbuconazole; trifloxystrobin; dimoxystrobin; epoxiconazole; fenpropidin; spiroxamine; boscalid; epoxiconazole; pyraclostrobin; trifloxystrobin; cyprodinil; pyraclostrobin; azoxystrobin; boscalid; bromuconazole; carbendazim; chlorothalonil; cyproconazole; cyprodinil; difenoconazole; dimoxystrobin; epoxiconazole; fenbuconazole; fenpropidin; fenpropimorph; fluoxastrobin; fluquinconazole; flusilazole; flutriafol; kresoxim-methyl; metconazole; metrafenone; picoxystrobin; prochloraz; propiconazole; prothioconazole; pyraclostrobin; quinoxyfen; spiroxamine; tebuconazole; tetraconazole; triadimenol; trifloxystrobin; and combinations thereof.

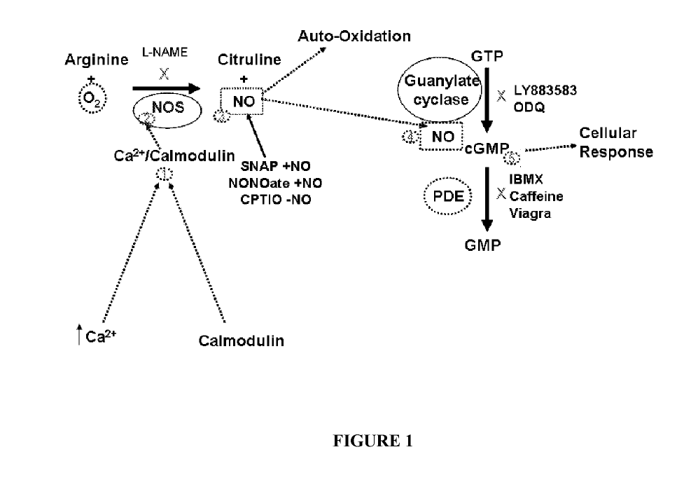

Nitric oxide (NO) signaling has some parallels to eATP signaling as Nitric oxide signaling in both plants and animals follows similar patterns. In plants, it appears that both nitric oxide synthase (NOS) and nitrate reductase (NR) are responsible for Nitric oxide production. In both systems increases in NO activate guanylate cyclase that uses GTP as a substrate to produce cGMP, which serves as a secondary messenger activating other cellular responses. The signal is diminished by phosphodiesterases that break down the cGMP into GMP as seen in the schematic of FIG. 1. Nitric oxide has been implicated in several plant processes such as: gravitropism (Hu et al., 2005), defense from pathogens, seed germination and de-etiolation (Beligni, 2000), ABA mediated stomatal closure (Bright et al., 2006), and flowering.

FIG. 1 illustrates some of the agonists and antogonists of the Nitric oxide signaling pathway that are affected or mediated via Nitric oxide. In FIG. 1 the Ca²⁺ binds to the calmodulin in 1 and in turn activates NOS in 2. The increase in NOS in 2 results in an increase in NO concentration that can be auto-oxidated or activates Guanylate cyclase in 4. The Guanylate cyclase can then convert GTP to cGMP in 5. Given the known inhibition of pollen germination by eATP (Steinebrunner et al., 2004) and the inhibition of pollen tube elongation by NO (Prado et al., 2004), it seemed likely that these two signaling mechanisms are interrelated in pollen germination and pollen tube growth, and initial tests appeared to confirm this relationship. Non-hydrolysable ATP and AMP analogs (ATPγS or AMPαS) were used along with agonists and antagonists of Nitric oxide signaling to study the affects on pollen germination and pollen tube growth. A Nitric oxide sensitive fluorescent dye (DAF-2D) was also used to visualize Nitric oxide production after nucleotide application. Nitric oxide signaling agonists lowered the concentration of ATPγS that inhibited pollen germination, and a Nitric oxide signaling antagonist was able to block the inhibitory action of ATPγS on both pollen germination and pollen tube growth. In elongating pollen tubes, an increase in Nitric oxide after application of ATPγS was visualized. It is clear that eATP inhibits pollen germination and pollen tube elongation by increasing Nitric oxide production that leads to increases in cGMP.

Effects of Extracellular ATPγS and AMPαS on Pollen Germination. Arabidopsis pollen was germinated in pollen germination media (PGM) containing from about zero to about 400 μM ATPγS or AMPαS. The addition of AMPαS did not significantly change the percent of germinated pollen. However, beginning at about 100 μM ATPγS the pollen germination rate significantly decreased, although, increasing concentrations of ATPγS did not show any greater decrease in germination. To provide a uniform sample that did not show great variation in concentration, a method of collecting a homogeneous population of pollen was developed. The method of collection included vortexing flowers in PGM creating a homogenous suspension of pollen from tens to hundreds of flowers. This method caused the release of a small amount of ATP, 2-5 μM into the PGM (data not shown). This level of ATP is several orders of magnitude lower than the 1-2 mM ATP that has been shown to affect pollen germination (Steinenbrunner et al., 2003), but it may induce down-regulation of the receptor as is known to be the case in animal and protist cells.

To determine if the affects of ATPγS on pollen germination was specific to Arabidopsis or a more generalized phenomenon, Medicago truncatula was also examined. Medicago also showed decreasing pollen germination rates starting with the application of 200 μM ATPγS.

The nitric oxide signaling pathway mediates the inhibition of Arabidopsis pollen germination by ATPγS. To examine the downstream effectors involved in the inhibition of Arabidopsis pollen germination by ATPγS, different agonists and antagonists of the known nitric oxide signaling pathway were examined. Each agonist or antagonist was used at a concentration that by itself did not change the percent of pollen germination. Some of the nitric oxide signaling pathway agonists are water soluble while others are DMSO soluble.

For water-soluble agonists: 50 μM ATPγS is not inhibitory and 100 μM ATPγS is inhibitory. For example, VIAGRA™ increases cGMP levels by inhibiting the phosphodiesterase that break down cGMP, NONOate is a nitric oxide donor and DibcGMP is a non-hydrolysable cGMP analog. Adding each of these chemicals activates the nitric oxide signaling pathway. Adding any one of these chemicals caused 50 μM ATPγS to become inhibitory to pollen germination.

For DMSO soluble chemicals, samples were all used at concentrations that resulted in a 0.1% final concentration of DMSO. With 0.1% DMSO 50 μM ATPγS inhibits and 100 μM ATPγS does not inhibit pollen germination. For example, IBMX increases cGMP levels by inhibiting the phosphodiesterase that break down cGMP and SNAP is a nitric oxide donor. Both of these chemicals added individually cause 50 μM ATPγS to inhibit pollen germination.

Production of cGMP and therefore downstream effects can be inhibited by ODQ, which inhibits guanylate cyclase. ODQ is DMSO soluble. The addition of 100 μM ODQ is able to reverse the inhibition of pollen germination by 250 μM ATPγS.

Even though inhibitors can have secondary or non-specific effects, five agonists of the nitric oxide signaling pathway, with different modes of action, lowered the concentration of ATPγS that inhibited pollen germination. In agreement with the agonist data, inhibiting cGMP production with ODQ reversed ATPγS inhibition of pollen germination. The consistency of these results demonstrates that the nitric oxide signaling pathway is activated in response to increased eATPγS.

Arabidopsis pollen tube elongation is inhibited by ATPγS. As vesicles fuse to deliver membranes to the tip of elongating pollen tubes, ATP is likely to be released. So ATPγS and AMPαS were investigated for their ability to affect pollen tube elongation. After pollen tubes had begun to grow, from about 0 to about 250 μM ATPγS or AMPαS were added and the subsequent growth rate was determined. Up to about 250 μM AMPαS had no significant effect on pollen tube elongation rates. However, more than 150 μM ATPγS significantly inhibited pollen tube elongation.

Blocking the nitric oxide signaling pathway reverses the inhibition of Arabidopsis elongation by ATPγS. The inhibition of Arabidopsis pollen tube elongation was also mediated via the NO signaling pathway. Pollen tube growth rates were measured in 0.5% DMSO with and without 100 μM ATPγS. The addition of 100 μM ODQ reversed the inhibition of pollen tube elongation by 100 μM ATPγS, while 100 μM ODQ had no effect alone, indicating that the NO signaling pathway is downstream of the inhibition of pollen tube elongation by ATPγS.

Increased extracellular ATPγS increases nitric oxide production in elongating pollen tubes. In addition to using agonists and antagonists to indirectly study the relationship of eATPγS and nitric oxide, direct measurements of increases in nitric oxide after application of ATPγS and AMPαS were examined. Pollen grains have a high level of auto-fluorescence and the nitric oxide levels in elongating Arabidopsis pollen tubes were determined using DAF-2D, a fluorescent marker for the presence of nitric oxide that was dissolved in DMSO. Elongating pollen tubes were treated with DAF-2D alone or with ATPγS or AMPαS. All pollen tubes showed fluorescence levels above background. Adding 125 μM AMPαS did not significantly change DAF-2D fluorescence, but the addition of 125 μM ATPγS significantly increased DAF-2D fluorescence. Thereby indicating that levels of ATPγS that inhibit pollen tube elongation also increase NO levels in elongating pollen tubes.

Growing Pollen tubes Release ATP into the Growth Media. During the first 12 minutes of growth of pollen tubes in germination media the [eATP] increases by about 26%, but if apyrase inhibitor is present in the media, the [eATP] increases by over 140%. Coincident with the increase in the [eATP] of the media containing apyrase inhibitor, the growth rate of the pollen tubes in this media decreases by over 33%; however, the fact that the tubes continue to grow in the medium containing apyrase inhibitor indicates that the accompanying increase in the media [eATP] cannot be attributed to the death of the tubes and makes it unlikely that their membranes are leaky, for tube growth requires maintenance of cell turgor. Both the increase in media [eATP] and the decrease in pollen tube growth rates are statistically significant (p<0.008).

The recent discovery that eATP induces a very rapid increase in [Ca²⁺]_{cyt} (Demichik et al., 2003; Jeter et al., 2004) raised the question of what downstream signaling steps followed from this central regulatory event. eATP inhibition of pollen germination, the nitric oxide inhibition of pollen tube elongation in lily, and the nitric oxide production is peroxisomes and calcium-dependent indicates nitric oxide is an intermediate signaling agent among the transduction events that mediate ATP responses in pollen.

ATPγS, like ATP, inhibits pollen germination, while ATPγS also inhibits pollen tube elongation. The effect of ATPγS concentration on NO production in pollen tubes was examined. Pollen tubes were examined rather than pollen grains because the tubes have far less autofluorescence than the grains, and the basal fluorescence with DAF-2D was above background. Addition of AMPαS did not change this fluorescence, but inhibitory levels of ATPγS significantly increased DAF-2D fluorescence.

To test whether the nitric oxide signaling pathway was mediating nucleotide effect on pollen germination and growth we used multiple agonists and antagonists of the NO signaling pathway. Although chemical mediators can have secondary or non-specific effects, six chemicals that impact the nitric oxide signaling pathway at several different points and that have different solubilities are very unlikely to have the same non-specific effects. Additionally, each chemical was used at a concentration that by itself had no effect on the pollen.

A key step by which nitric oxide induces responses in plants and animals is by the activation of guanylate cyclase and subsequent production of cGMP. The concentration of eATP that induce nitric oxide production likely have their effects on pollen germination and growth by inducing the production of cGMP via guanylate cyclase. Evidence for this is that the concentration of ATPγS that inhibits pollen germination is lower in the presence of agonists of the nitric oxide signaling pathway. Additionally, when ODQ, an inhibitor of guanylate cyclase, was applied, the inhibition of pollen germination by ATPγS was reversed.

One assumption might be that the inhibition of pollen germination by ATPγS is due to the lack of pollen tube elongation. The fact that pollen germination and pollen tube growth show different sensitivities to ATPγS is not consistent with this conclusion. Pollen tube elongation is less sensitive to ATPγS than pollen germination. The responses to inhibitory levels of ATPγS are similar, but the levels of ATPγS needed for inhibition are different in pollen germination and elongating pollen tubes. The difference in perception may have to do with receptor sensitivity or endogenous levels of eATP.

The cytoplasm contains low mM concentrations of ATP, and among the several mechanisms that have been proposed for the release of this ATP to the exterior, one involves the fusion of secretory vesicles, typically enclosing high levels of ATP, to the plasma membrane. Pollen tube elongation involves massive amounts of vesicle fusion at the growing tip and so could be accompanied by significant release of ATP to the exterior of the cell.

Changes in [eATP] may be perceived by different mechanisms. In animals, the binding of eATP to P2 receptors has been well-described. Other mechanisms of eATP perception may also include perturbation of the steep concentration gradient between the cytoplasmic and ECM concentrations of ATP (Thomas). P2-like receptors have not been identified in plants, and the sequence divergence of animal P2 receptors has made searching for plant analogs difficult (Jeter et al., 2004).

Both NOS and NR have been identified as being sources of nitric oxide in plants and both are potentially regulated by calcium (Huber et al., 1996; Corpas et al., 2004). If nitric oxide is part of a signal transduction chain in pollen, then some nitric oxide producing enzymes must exist in pollen. Two recent studies have looked at microarray data from Arabidopsis pollen. Honys and Twell (2004) only show AtNOS1 expression in uninucleate microspores, but do show NR1 and NR 2 expression in trinucleate pollen. Yet they did not detect AtNOS1 or NR1 or 2 in mature pollen grains. Conversely Pina et al., (2005) did detect AtNOS1 and NR1 and 2 in pollen. Both of these studies quantified mRNA levels, which may or may not reflect changes in protein levels.

Normally, any ATP released during growth would be expected to be hydrolyzed rapidly by the high levels of acid phosphatases and ectoapyrases expressed by pollen tubes. Previous findings showed that Arabidopsis pollen lacking both the ATP hydrolyzing enzymes apyrase 1 and apyrase 2 fail to germinate (Steinebrunner et al., 2003). Exactly how these apyrases mediate pollen germination is still unknown, but one possible explanation is that they are needed to lower eATP levels prior to pollen germination.

The responses of pollen to ATPγS require the same 100-200 μM concentrations of ATPγS as needed to induce increases in [Ca²⁻]_{cyt} in Arabidopsis seedlings (Jeter et al., 2004), and that are commonly used to activate P2X purinoceptors in animal cells, but considerably more than the 250 nM needed to induce increases in [Ca²⁺]_{cyt} in Arabidopsis roots (Demidchik et al., 2003) and the production of reactive oxygen species in leaves (Song et al., 2006). One obvious explanation for this could be that different tissues have different sensitivities to eATP, just like roots and shoots have different sensitivities to auxin. Alternatively, the native sensitivity of pollen to eATP could be reduced due to the down-regulation of the ATP receptor by an initial ATP exposure, as is known to occur in animals and protists. The initial ATP exposure could occur in vivo (e.g., by cellular ATP released during the programmed cell death of the surrounding tapetal layer in anthers as pollen matures) or during pollen isolation (there is 2 to 5 μM ATP in the pollen/PGM suspension after vortexing the flowers to release pollen). Of course, the level of ATPγS applied to pollen in the bulk medium does not predict how much of it reaches the plasma membrane, where animal purinoceptors function. The 100-200 μM nucleotide may be “physiological” relevant depending on the [eATP] in the zone immediately exterior to the plasma membrane. As a comparison, animal cells in this zone have significantly higher [ATP] than more peripheral zones of the ECM.

ATPγS is acidic and can bind divalent cations, but the pH of the PGM medium used for pollen germination and pollen growth assays was not altered by the concentrations of ATPγS tested, and the chelating capacity of ATPγS was negligible compared to the concentrations of divalent cations present in PGM. Data interpretation was also not complicated by the release of phosphate from the applied nucleotides during the treatment period, because although cells can hydrolyze eATP with ectoapyrases and ectophosphatases, neither of these enzymes can use ATPγS as a substrate. Nucleotide effects on Arabidopsis pollen are not species specific, as similar germination and growth responses are seen in pollen of Medicago truncatula. Finally, in parallel with earlier studies on other nucleotide-induced responses in plants, ADPβS is an active agonist of pollen responses and AMPαS is not (Jeter et al., 2004; Song et al., 2006).

While the literature regarding nitric oxide signaling in plants is well established, the idea of eATP signaling is still developing. eATP is a physiological signal in plants by elucidating some part of its signal transduction. Pollen served as a model system because of the already established role of nitric oxide in pollen tube elongation (Prado et al., 2004) and the previously identified inhibition of pollen germination by ATP (Steinebrunner et al., 2003). Results from testing several mediators of nitric oxide signaling and measurements of nitric oxide levels support the conclusion that an increase in eATP leads to inhibition of pollen germination and pollen tube elongation in part because these signals are transduced via nitric oxide and cGMP. These results increase the known cellular changes induced by eATP, and are the first report of a connection between eATP and NO.

Strains and Growth Conditions. For the experiments related to the nitric oxide-ATP signaling relationship, all Arabidopsis thaliana plants were ecotype Wassilewskija (WS), and Medicago truncatula plants were strain A17. All of the plants were grown in Metro-Mix 200 soil at 24° C. with constant light. In vitro Pollen Germination. Arabidopsis flowers in stage 12-14 (Smyth et al., 1990) or Medicago truncatula flowers in stage F3 (Firnhaber et al., 2005) were collected and vortexed for 1 min in pollen germination media (PGM) (0.01% boric acid, 1 mM MgSO₄, 1 mM CaCl₂, 1 mM Ca(NO₃)₂, 5 mM HEPES, 18% Sucrose pH 7.0). The supernatant containing the pollen was collected by pipet, and up to three rounds of vortexing of the same pollen could be pooled together. The pollen suspended in PGM was then added in 10-12 μL drops to 400 μL PGM+1% agar that had been spread as a thin film across a microscope slide. The slides and pollen were then placed into humidity chambers, petri dishes with water saturated kimwipes, and allowed to germinate at 26° C. overnight. Pictures of the germinated pollen were taken with a digital camera attached to a brightfield microscope at 40×. The digital images were scored for the percent of pollen germinated using the AlphaEase software from Alpha Innotech. Pollen with tubes that were shorter than the diameter of the pollen grain were not counted as germinated or non-germinated. The chemicals: ATPγS, AMPαS, N2, 2-O-dibutyrylguanosine 3′:5′-cyclic monophospohate (Dib cGMP), Sildenafil citrate (Viagra™, Pfizer), and 2-(N,N-Diethylamino)-diazenolate 2-oxide (NONOate) were added to the pollen/PGM suspension prior to adding the drops to the PGM+1% agar. The chemicals 1H-[1,2,4]Oxadiazolo[4,3,-a]quinoxalin-1-one (ODQ), N-[4-[1-(3-Aminopropyl)-2-hydroxy-2-nitrosohydrazino]butyl]-1,3-propanediamine (SNAP), and 3-isobutyl-1-methylxanthine (IBMX) were diluted in DMSO and added to the pollen/PGM suspension to a final concentration of DMSO of 0.1%.

For successful pollen germination it was critical for the plants to be healthy and growing in slightly dry soil. To achieve this, the plants were briefly watered 1-2 days before the experiment. As Johnson-Brousseau and McCormick note in their paper (Johnson-Brousseau and McCormick 2004), a solid base is necessary for best in vitro germination of Arabidopsis pollen. The pollen would not germinate in liquid PGM if not overlaid on a solid PGM+1% agar base, and the vortexing and deposition of the pollen/PGM suspension needed to take place in less than 30 minutes or germination rates dropped considerably.

Measurements of pollen tube elongation. Pollen was germinated as described above except 10 μL drops of pollen/PGM suspension were added to 60 μL of PGM+1% agar that had been deposited into a concavity slide. The pollen was allowed to germinate for 1-2 h, so that pollen tubes were clearly visible. Then 90 μL of PGM or PGM containing ATPγS, AMPαS, DMSO or ODQ was added and a photo was taken using a brightfield microscope at 40×. The coordinates of each photo were noted, so that the exact pollen tubes could be photographed 50-60 minutes later. Using Image J, the pollen tube lengths for individual pollen tubes were measured and a growth rate of μm/min was determined.

Measurements of NO in growing pollen tubes treated with ATPγS or AMPαS. Pollen was germinated as described for the pollen elongation measurements. After 1-2 hours when pollen tubes were clearly visible, 90 μL of PGM containing 5 μM 4,5-diaminofluorescein diacetate (DAF-2D, Molecular Probes) with or without ATPγS or AMPαS was added. Using a confocal laser microscope (Leica SP2 AOBS) with emissions at 488 nm and a filter at 522 nm optical sections taken of several pollen tubes from 5-30 min after addition of the DAF-2D and nucleotides. Using Image J, the average fluorescence for the pollen tubes on each slide and each time point were determined. Since background fluorescence for each slide varied, three areas where no pollen tubes were present were averaged and subtracted from the pollen tube averages for each slide. Pollen tubes with and without nucleotides were compared to each other at similar time points to determine if there was a difference in fluorescence.

ATP Assay. The luciferin-luciferase assay to determine ATP release was performed using an ATP bioluminescent assay kit (Sigma-Aldrich). After flowers were vortexed, the pollen/PGM solution was filtered through a 0.45 μm filter to remove the pollen, and then the filtered solution was immediately frozen in liquid N₂. To measure the released ATP in the solution, each solution was added into the bioluminescent assay solution. The luminescence signal was integrated for 1 minute.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations can be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Burnstock G and Knight G E (2004) Cellular distribution and functions of P2 receptor subtypes in different systems. Int Rev Cytol 240: 31-51.

2. Jaffe M J (1973) The role of ATP in mechanically stimulated rapid closure of the Venus's-Flytrap. Plant Physiol. 51: 17-18.

3. Williamson R E (1975) Cytoplasmic streaming in chara: A cell model activated by ATP and inhibited by cytochalasin B. J Cell Sci 17: 655-668.

4. Nejidat A, Itai C, Roth-Bejerano N (1983) Stomatal response to ATP mediated by phytochrome. Physiol Plant 57, 367-370.

5. Kamizyo A, Tanaka N (1982) Studies on the generative nuclear divisions. III. Effects of exogenous ATP on the generative nuclear divisions in Lilium longiflorum. Cytologia 47: 195-205.

6. Demidchik V, Nichols C, Oliynyk M, Dark A, Glover, B J, Davies J M (2003) Is ATP a signaling agent in plants? Plant Physiol 133: 456-461.

7. Burnstock G (1972) Purinergic nerves. Pharmacol Rev 24: 509-581.

8. Burnstock G (1996). Purinoceptors: Ontogeny and phylogeny. Drug Dev. Res. 39: 204-242.

9. Lew R R, Dearnaley J D W (2000) Extracellular nucleotide effects on the electrical properties of growing Arabidopsis thaliana root hairs. Plant Science 153: 1-6.

10. Jeter C, Tang W, Henaff E, Butterfield T, Roux S J (2004) Evidence of a novel cell signaling role for extracellular adenosine triphosphates and diphosphates in Arabidopsis. Plant Cell 16: 2652-2664.

11. Arteca, J, Arteca N (1999) A multi-responsive gene encoding 1-aminocyclopropane-1-carboxylate synthase (ACS6) in mature Arabidopsis leaves. Plant Mol Biol 39: 209-219.

12. Sauer H, Hescheler J, Wartenberg M (2000) Mechanical strain-induced Ca2+ waves are propagated via ATP release and purinergic receptor activation. Am J Physiol Cell Physiol 279: C295-C307.

13. Song C (2004) Extracellular ATP signaling: induction of superoxide accumulatin and possible regulation by ectopayrases in Arabidopsis thaliana. PhD dissertation, The University of Texas at Austin.

14. Song C, Stout S, Roux S J (2004) Extracellular nucleotide signaling induces the accumulation of superoxide in Arabidopsis thaliana. Plant Biology 2004, Abstract #450, URL: http://216.133.76.127/pb2004/public/P51/7845.html.

15. Jabs T, Dietrich R A, Dangl J L (1996) Initiation of runaway cell death in an Arabidopsis mutant by extracellular superoxide. Science 273 1853-1856.

16. Mahalingam R, Fedoroff N (2003) Stress response, cell death and signalling: the many faces of reactive oxygen species. Physiol Plant 199: 56-68; Liszkay A, van der Zalm E, Schopfer P (2004) Production of reactive oxygen intermediates (O-2(center dot-), H2O2, and (OH)—O-center dot) by maize roots and their role in wall loosening and elongation growth. Plant Physiol 136: 3114-3123.

17. Vignais P V (2002) The superoxide-generating NADPH oxidase: Structural aspects and activation mechanism. Cell Mol Life Sci 59: 1428-1459.

18. Mittler R, Vanderauwera S, Gollery M, Van Breusegem F (2004) Reactive oxygen gene network of plants. Trends in Plant Sci 9: 490-498.

19. Tang W Q (2004) Effects of extracellular ATP and ADP on growth and development of Arabidopsis seedlings. PhD dissertation, The University of Texas at Austin.

20. Tang W Q, Brady S R, Sun Y, Muday G K, Roux S J (2003) Extracellular ATP inhibits root gravitropism at concentrations that inhibit polar auxin transport. Plant Physiol 131: 147-154.

21. Noh B, Bandyopadhyay A, Peter W, Spalding E, Murphy A (2003) Enhanced gravi- and phototropism in plant mdr mutants mislocalizing the auxin efflux protein PIN1. Nature 423: 999-1002.

22. Schopfer P (2001) Hydroxyl radical-induced cell-wall loosening in vitro and in vivo: implications for the control of elongation growth. Plant J 28: 679-688.

23. Coco S, Calegari F, Pravettoni E, Pozzi D, Taverna E, Rosa P, Matteoli M, Verderio C (2003) Storage and release of ATP from astrocytes in culture. J Biol Chem 278: 1354-1362

24. Clark G, Lee D, Dauwalder M, and Roux S J (2005) Immunolocalization and histochemical evidence for the association of two different Arabidopsis annexins with secretion during early seedling growth and development. Planta 220: 621-631.

25. Steinebrunner I, Wu J, Sun Y, Corbett A, Roux S J (2003) Disruption of apyrases inhibits pollen germination in Arabidopsis. Plant Physiol 131: 1638-1647.

26. Cohn J R, Uhm T, Ramu S, Nam Y-W, Kim D-J, Penmetsa V, Wood T C, Denny R L, Young N D, Cook, D R, Stacey G (2001) Differential regulation of a family of apyrase genes from Medicago truncatula. Plant Physiol 125: 2104-2119.

27. Kawahara T, Toyoda K, Kiba A, Miura A, Ohgawara T, Yamamoto M, Inagaki Y, Ichinose Y, and Shiraishi T (2003) Cloning and characterization of pea apyrase: involvement of PsAPY1 in response to signal molecules from the pea pathogen. Mycosphaerella pinodes. J Gen Plant Pathol 69: 33-38.

28. Bonsignore C A, Hepler P K. Caffeine inhibition of cytokinesis: Dynamics of cell plate formation-deformation in vivo. Protoplasma 1985; 129: 28-35.

29. Roberts A W, Haigler C H. Methylxanthines reversibly inhibit tracheary element differentiation in suspension cultures of Zinnia elegans. Planta 1992; 186, 586-592.

30. Pierson E S, Smith P J S, Shipley A M, Jaffe L F, Cresti M, Hepler P K. Ca2+ fluxes around pollen grains and pollen tubes of lily; normal development and effect of temperature shock, BAPTA-type buffer microinjection and depletion of boric acid from the medium. Biol. Bull. 1993; 185: 302-303.

31. Brault M, Caiveau O, Pedron J, Maldiney R, Sotta B, Miginiac E. Detection of membrane-bound cytokinin-binding proteins in Arabidopsis thaliana cells. Eur J Biochem 1999; 260: 512-9.

32. Dubyak G R, el-Moatassim C. Signal transduction via P2-purinergic receptors for extracellular ATP and other nucleotides. Am J Physiol 1993; 265: C577-606.

33. Sauer H, Hescheler J, Wartenberg M. Mechanical strain-induced Ca2+ waves are propagated via ATP release and purinergic receptor activation. Am J Physiol Cell Physiol 2000; 279: C295-C307.

34. Bowler W B, Buckley K A, Gartland A, Hipskind R A, Bilbe G, Gallagher J A.

Extracellular nucleotide signaling: a mechanism for integrating local and systemic responses in the activation of bone remodeling. Bone 2001; 28: 507-12.

35. Boyum R, Guidotti G. Effect of ATP binding cassette/multidrug resistance proteins on ATP efflux of Saccharomyces cerevisiae. Biochem Biophys Res Commun 1997; 230: 22-6.

36. Thomas C, Rajagopal A, Windsor B, Dudler R, Lloyd A, Roux S J. A role for ectophosphatase in xenobiotic resistance. Plant Cell 2000; 12: 519-533.

37. Handa M, Guidotti G. Purification and cloning of a soluble ATPdiphosphohydrolase (apyrase) from potato tubers (Solanum tuberosum). Biochem Biophys Res Commun 1996; 218: 916-23.

38. Hsieh H, Tong C, Thomas C, Roux S J. Light-modulated abundance of an mRNA encoding a calmodulin-regulated, chromatin-associated NTPase in pea. Plant Mol Biol 1996; 30:135-147.

39. Roberts N J, Brigham J, Wu B, Murphy J B, Volpin H, Phillips D A, Etzler M E. A nod factor-binding lectin is a member of a distinct class of apyrases that may be unique to the legumes. Mol Gen Genet 1999; 262: 261-267.

40. Steinebrunner I, Jeter C, Song C, Roux S J. Molecular and biochemical comparison of two different apyrases from Arabidopsis thaliana. Plant Physiol Biochem 2000; 38: 1-10.

41. Day R B, McAlvin C B, Loh J T. Denny R L, Wood T C, Young N D, Stacey G. Differential expression of two soybean apyrases, one of which is an early nodulin. Mol Plant Microbe Interact 2000; 13, 1053-1070.

42. Cohn J R, Uhm T, Ramu S, Nam Y-W, Kim D-J, Penmetsa V, Wood T C, Denny R L, Young N D, Cook, D R, Stacey G. Differential regulation of a family of apyrase genes from Medicago truncatula. Plant Physiol 2001; 125: 2104-2119.

43. Thomas C, Sun Y, Naus K, Lloyd A, Roux S J. Apyrase functions in plant phosphate nutrition and mobilizes phosphate from extracellular ATP. Plant Physiol 1999; 119: 543-551.

44. Steinebrunner I, Wu J, Sun Y, Corbett A, Roux S J. Disruption of apyrases inhibits pollen germination in Arabidopsis. Plant Physiol 2003; 131: 1638-1647.

45. Kalsi G, Etzler M E. Localization of a Nod factor-binding protein in legume roots and factors influencing its distribution and expression. Plant Physiol 2000; 124: 1039-48.

46. McAlvin C B, Stacey G. Transgenic expression of the soybean apyrase in Lotus japonicus enhances nodulation. Plant Physiol 2005; 137: 1456-62.

What is claimed is:

1. A composition that modulates drug resistance in a plant comprising:
one or more extracellular nucleotides that contact a plant cell and modulate drug resistance in the plant cell.

2. The composition of claim 1, wherein the one or more extracellular nucleotides comprises ATP, ADP, UTP, UDP, CTP, CDP, TTP, TDP, GTP, GDP, dATP, dADP, dUTP, dUDP, dCTP, dCDP, dTTP, dTDP, dGTP, dGDP, stable analogues and combinations thereof.

3. The composition of claim 1, wherein the one or more extracellular nucleotides increase the concentration of one or more reactive oxygen species, nitric oxide or both within the plant cell.

4. The composition of claim 3, wherein the reactive oxygen species comprise superoxide, hydrogen peroxide, hydroxyl radical, singlet oxygen, hypochlorous acid and combinations thereof.

5. The composition of claim 1, wherein the one or more extracellular nucleotides alter the transcription of one or more genes selected from a ERF2 gene, a ERF3 gene, a ERF4 gene, a PAL1 gene, a LOX2 gene and a ACS6 gene.

6. A composition that modulates drug resistance in a plant comprising:
one or more herbicides; and
one or more extracellular nucleotides that contact a plant cell to affect the plant's drug resistance.

7. The composition of claim 6, wherein the one or more extracellular nucleotides comprise ATP, ADP, UTP, UDP, CTP, CDP, TTP, TDP, GTP, GDP, dATP, dADP, dUTP, dUDP, dCTP, dCDP, dTTP, dTDP, dGTP, dGDP and stable analogues and combinations thereof.

8. The composition of claim 6, wherein the one or more extracellular nucleotides increase the concentration of a reactive oxygen species, superoxides, NO or a combination thereof.

9. The composition of claim 8, wherein the reactive oxygen species comprise superoxide, hydrogen peroxide, hydroxyl radical, singlet oxygen, hypochlorous acid and combinations thereof.

10. The composition of claim 6, wherein the one or more herbicides, hormones, fungicides or a combination thereof.

11. A method of altering the resistance of a plant to a herbicide comprising the steps of:
increasing the concentration of one or more extracellular nucleotides about a plant cell to modulate drug resistance of the plant.

12. The method of claim 11, wherein the one or more extracellular nucleotides comprise ATP, ADP, UTP, UDP, CTP, CDP, TTP, TDP, GTP, GDP, dATP, dADP, dUTP, dUDP, dCTP, dCDP, dTTP, dTDP, dGTP, dGDP, and stable analogues and combinations thereof.

13. The method of claim 11, wherein the one or more extracellular nucleotides affect one or more stress related biosynthetic genes of the plant.

14. The method of claim 11, wherein the one or more extracellular nucleotides affect one or more stress related biosynthetic genes selected from a ERF2 gene, a ERF3 gene, a ERF4 gene, a PAL1 gene, a LOX2 gene, a ACS6 gene and combinations thereof.

15. The method of claim 11, wherein the one or more extracellular nucleotides affect the concentration of superoxide, NO, hydrogen peroxide, hydroxyl radical, singlet oxygen, hypochlorous acid or a combination thereof.

16. The method of claim 11, wherein the one or more extracellular nucleotides are not degradable by extracellular enzymes.

17. A method of increasing the herbicidal sensitivity of a plant comprising:
increasing the concentration of one or more extracellular nucleotides about the plant cell to affect the herbicidal sensitivity of the plant.

18. The method of claim 17, wherein the one or more extracellular nucleotides comprises ATP, ADP, UTP, UDP, CTP, CDP, TTP, TDP, GTP, GDP, dATP, dADP, dUTP, dUDP, dCTP, dCDP, dTTP, dTDP, dGTP, dGDP and stable analogues and combinations thereof.

19. The method of claim 17, wherein the one or more extracellular nucleotides affect the concentration of superoxide, NO, hydrogen peroxide, hydroxyl radical, singlet oxygen, hypochlorous acid and combinations thereof.

20. The method of claim 17, wherein the one or more extracellular nucleotides modulate the level of transcription of one or more biosynthetic genes.

21. A herbicide potentiator that modulates drug resistance in a plant comprising:
one or more extracellular nucleotides that contact a plant cell membrane and modulate drug resistance.

22. The composition of claim 21, wherein the one or more extracellular nucleotides comprises ATP, ADP, UTP, UDP, CTP, CDP, TTP, TDP, GTP, GDP, dATP, dADP, dUTP, dUDP, dCTP, dCDP, dTTP, dTDP, dGTP, dGDP, and stable analogues and combinations thereof.

23. The composition of claim 21, wherein the one or more extracellular nucleotides increase the concentration of reactive oxygen species, NO or both.
